(19) United States
(12) Patent Application Publication
(10) Pub. No.: US 2008/0113390 A1
(43) Pub. Date: May 15, 2008

(54) Personalized assay for the identification of an effective therapy for cancer

(76) Inventors: In Suk Han, Sandy, UT (US); Young Hee Ko, Owings Mills, MD (US)

Correspondence Address: THORPE NORTH & WESTERN, LLP., P.O. Box 1219, SANDY, UT, 84091-1219, US

(21) Appl. No.: 11/891,315

(22) Filed: Aug. 8, 2007

Related U.S. Application Data
(60) Provisional application No. 60/836,553, filed on Aug. 8, 2006.

(51) Int. Cl. G01N 33/574; C12Q 1/54

(52) U.S. Cl. 435/7.23; 435/14

(57) ABSTRACT

Effective anticancer agents for use in treatment of particular cancers can be predicted by testing whether particular anticancer agents are effective in reducing the lactic acid production by sample cells removed from the particular cancerous tissue to be treated. Similarly, effective anticancer agents can be predicted by testing whether particular anticancer agents are effective in reducing ATP levels in sample cells removed from the particular cancerous tissue. A method of predicting anticancer agents that will be effective for use in treating a particular cancer includes the steps of obtaining a plurality of samples of the cancerous tissue to be treated and determining the capacity of a sample to produce lactic acid in both the presence and absence of a particular anticancer agent. Anticancer agents that most reduce the lactic acid production should be effective treatment agents. Similarly, anticancer agents that most reduce the presence of ATP in the sample cancer cells should be effective treatment agents.

### RELATED APPLICATIONS

This application claims the benefit of Provisional Application Ser. No. 60/836,553, filed Aug. 8, 2006, and entitled “Personalized Assay For The Identification Of An Effective Therapy For Cancer”.

### BACKGROUND OF THE INVENTION

1. Field

The invention is in the field of cancer treatment.

2. State of the Art

The number of people in the world who are likely to acquire cancer in the future is staggering, and it is predicted by some reports that as many as 1 in 2 men and 1 in 3 women will die painfully of this disease. In addition, there are many animals, particularly pets, that become cancer victims and die of the disease. Unlike many bacterial infections that are controllable by antibiotics like penicillin or derivatives thereof, there is to date no “penicillin” for cancer. Rather, there are many chemical agents used for treating cancer, but a chemical agent that might be effective for one type of cancer will have no effect on another type. This is in part due to the heterogeneous nature of cancer cells. Therefore, a very real and immediate need exists for more personalized methods to decide quickly after a cancer diagnosis which of the currently available anticancer agents to prescribe for any given cancer patient (human or animal).

Significantly, one of the most commonly used diagnostic tests for clinical cancers used worldwide is positron emission tomography (PET). This diagnostic method identifies those cancers (the vast majority of all cancers) that exhibit an enhanced utilization of glucose. This common tumor metabolic phenotype results in the enhanced catabolism (breakdown) of glucose and therefore the enhanced rate of production of energy in the form of adenosine triphosphate (ATP) that fuels directly tumor growth. It has long been known via studies in experimental animals that those cancers that are the most advanced (i.e., poorly differentiated) exhibit the highest glucose catabolic rate (conversion of glucose to lactic acid) and almost invariably lead to the death of their host. Therefore, it is of the utmost urgency that the treatment of patients whose cancers exhibit this phenotype, as revealed by PET, obtain as quickly as possible an effective chemotherapeutic agent that inhibits the conversion of glucose to lactic acid. This assures the destruction of much of the ATP production machinery and the subsequent death or growth arrest of the tumor.

### SUMMARY OF THE INVENTION

The personalized cancer therapy assay of the invention is designed to individualize choices made in selecting an anticancer agent for the treatment of any cancer patient harboring one or more PET positive tumors. The assay can be used to screen up to 24 or more known anticancer agents at any one time. Moreover it is sufficiently rapid that once a fresh biopsy of the tumor has been obtained the physician/oncologist responsible for treating the patient should have an answer as to which anticancer agent or agents to employ as therapy within no more than 3-4 days, if not sooner.

Included in the assay will be glucose, cofactors for glucose catabolism, and tiny unfrozen, fresh samples obtained directly from a biopsy of the original tumor that has been diagnosed clinically as PET positive, i.e., as utilizing/metabolizing enhanced amounts of glucose. Specifically, the end product of glucose metabolism, lactic acid, will be monitored in the absence and in the presence of known and widely used anticancer agents as well as those recently approved for clinical trials, and those soon to become approved. In addition to monitoring lactic acid, cellular ATP content will be monitored in a separate set of experiments.

The invention provides a method of predicting effective anticancer agents for treating a particular cancer in a patient which includes the step of obtaining a plurality of samples of the cancerous tissue from the particular cancer to be treated in a patient. A determination of the capacity of each tissue sample to convert glucose to lactic acid is made. Then, a determination of the capacity of a tissue sample to convert glucose to lactic acid in the presence of a particular anticancer agent is made. The particular anticancer agents that most inhibit the capacity of a sample to convert glucose to lactic acid are the anticancer agents indicated as the most promising as effective anticancer agents to administer to the patient for treatment of the particular cancer. Generally, once the most promising anticancer agents are determined by determining the reduction in lactic acid production, the sample cells are tested to determine the reduction in ATP level caused by the anticancer agents. Those anticancer agents causing the most reduction in lactic acid production and the most ATP reduction are indicated as the most promising as effective anticancer agents to administer to the patient.

The most unique feature of this invention is that it personalizes the choice by which an anticancer agent is selected to treat a cancer patient. A second unique feature is the invention utilizes fresh unfrozen, tumor tissue obtained directly from a patient's cancerous tumor. This avoids a process of generating the corresponding tumor cell line, which can change the cancer phenotype by introducing further mutations, and therefore shortens the time for identifying one or more effective anticancer agents. A third feature of the invention is that it takes advantage of one of the most common methods in clinics throughout the world to diagnose cancer (PET scans). Finally, the invention will allow the physician/oncologist who has diagnosed the cancer to advise the patient as soon as possible about available agents that might be successful in treating her/his cancer.

### DETAILED DESCRIPTION OF AN EMBODIMENT OF THE INVENTION

The invention provides a test or assay for determining which of numerous anticancer agents will be the most effective in treating a particular cancer to be treated. The assay is effective for use with PET positive cancers. PET positive cancers exhibit an enhanced utilization of glucose. This common tumor metabolic phenotype results in the enhanced catabolism of glucose (conversion of glucose to lactic acid) and therefore the enhanced rate of production of energy in the form of adenosine triphosphate (ATP) that fuels directly tumor growth. The effectiveness of any particular anticancer agent against the particular cancer to be treated can be determined by measuring the effectiveness of the agent in stopping or slowing the catabolism of glucose and the production of ATP by the particular cancer cells to be treated. One way to determine the effectiveness of an agent in stopping or slowing the catabolism of glucose is to measure the effectiveness of the agent in stopping or slowing the production of lactic acid by the cancer cell. Thus, the assay will monitor the production of lactic acid by the cancer cell in the environment of the body, this body environment will be simulated for the test, and the production of lactic acid by the cancer cell in the presence of known or suspected anticancer agents. Those agents that stop or show the greatest slowing of the lactic acid production are the agents that should be most effective in killing the particular cancer cells to be treated. In addition, the cellular ATP content of the cancer cells to be treated can be measured both in the environment (simulated) of the body and in the presence of the known or suspected anticancer agents. Those anticancer agents that most reduce the ATP content of the cancer cells should be the most effective agents in killing the particular cancer cells to be treated. In one embodiment of the assay of the invention, the agents that stop or most slow the lactic acid production of the cancer cells are determined and, where there are several agents indicated as being effective in stopping or slowing the lactic acid production, the cancer cells are tested with those agents to determine which are most effective in reducing ATP content of the cancer cells. Those agents which most reduce the ATP content of the cancer cells are then indicated as the most effective agents to use in treatment of the cancer to be treated.

In one embodiment of the assay of the invention, fresh tumor material will be obtained from a consenting patient via one or more approved procedures for obtaining a tumor biopsy. The tumor material will be placed immediately in a medium on ice and maintained on ice (or at a cold temperature, near 4° C.). Very small identical size samples will be excised from the cold tumor biopsy material using an available cutting device. Samples near a size of 1 mm×1 mm×1 mm, or if necessary another size, will be obtained. Each sample will be placed in one of the wells of, for example, a 96 well plate containing a glucose cell culture medium that will support the viability of the tissue at 37° C. The choice of the medium may vary but each should contain glucose, fetal bovine serum, and other essential nutrients to keep the tissue viable. This simulates the body environment for the cells. After various time intervals aliquots of the medium surrounding each of the identical size biopsy samples will be removed to determine the amount of glucose that has been converted to lactic acid, and therefore establish the basal rate of glucose catabolism (breakdown) for each sample. (It is expected that for each sample this basal rate will be very nearly the same, although small variations are possible). Assays for determining lactic acid can be based on 1) NADH production by lactate dehydrogenase, 2) H₂O₂ production by lactate oxidase, or 3) a change in pH.

Once the basal rate of glucose catabolism has been established for each sample derived from the tumor biopsy, anticancer agents that could be used to treat the cancer to be treated will be added to wells containing a biopsy sample to determine the agent's capacity to inhibit the rate of lactic acid production from glucose. The anticancer agents tested can be approved known anticancer agents, soon to be approved anticancer agents, in the “pipeline” for consideration as an anticancer agent, or other agents desired to be tested for possible effectiveness. The amount of a particular anticancer agent added to a well will generally be based on the known or generally accepted therapeutic amount used for cancer treatment for that particular agent. The wells not containing tumor tissue will serve as controls for measuring the extent of interference (if any) by the anticancer agents in determining lactic acid. Aliquots from control wells and those wells containing the tumor tissue samples will be removed at different time points and each will be subjected to determination of lactic acid. Of the anticancer agents tested, those that inhibit lactic acid production completely or nearly completely will be considered as promising candidates to administer to the patient as cancer therapy. However, prior to making recommendations, ATP levels in the cells can be tested as those anticancer agents that inhibit lactate production completely, or nearly completely, would be predicted to markedly reduce ATP levels. ATP levels can be determined by luminometry using the firefly assay that includes luciferin and luciferase. Those anticancer agents tested that inhibit lactic acid production and also reduce the ATP levels can then be recommended for use in treatment of the cancer to be treated.

Where several different anticancer agents are recommended for use in treatment of the cancer to be treated as a result of the assay of the invention, the final determination of which agent to use for the actual treatment can consider other factors such as known side effects of a particular recommended anticancer agent and availability and cost of a particular recommended anticancer agent.

The test of the invention, in addition to indicating particular agents that should be most effective in treating a particular cancer, can also indicate a cancer's resistance to particular anticancer agents. A lack of the reduction in lactic acid production or a lack of reduction in the ATP levels indicate a resistance of the particular cancer to the anticancer agent.

The test or assay of the invention allows personalizing the choice of cancer therapy for any patient (human or animal) suffering from a cancerous tumor that is diagnosed clinically to exhibit a positive positron emission tomography scan (PET scan). The test utilizes fresh tumor samples obtained from a patient via biopsies or surgery, and can utilize any size sample that can be obtained. Preferably, the sample obtained from the patient is large enough to be subdivided, such as by use of a cutting device, to provide a plurality of smaller individual samples for testing of different anticancer agents. Individual samples of 1 mm×1 mm×1 mm have been found satisfactory for testing although other sizes can be used, such as samples ranging from 0.01 mm×0.01 mm×0.01 mm to 20 mm×20 mm×20 mm each in size. The individual samples should be of substantially the same size and are each placed in a sample well of a well plate having a plurality of sample wells. Well plates having 96 sample wells are commonly commercially available. The cutting device used to subdivide the biopsy sample for the assay can be designed to cut the same size individual samples and to remove these samples from the biopsy sample and discharge them directly into the sample wells.

The sample wells containing samples will also contain a cell culture medium for maintaining the viability of the sample cells. Various cell culture mediums or media can be used, and generally a glucose cell culture medium will be used. The cell culture medium will generally be maintained at a temperature between about 4° C. and 45° C. The cell samples will be tested for their capacity to convert glucose to lactic acid both in the absence and the presence of anticancer agents. After testing for production of lactic acid in the absence of an anticancer agent, an anticancer agent can be added to the sample well so production of lactic acid in the presence of the anticancer agent can be measured. The concentration of a particular anticancer agent added to a well will generally be based on the known or generally accepted therapeutic amount used for cancer treatment for that particular agent. The cell content of ATP will be measured prior to and after the addition of an anticancer agent for those cases where the anticancer agent has been shown to inhibit the conversion of glucose to lactic acid. This further assures that the anticancer agent selected will be therapeutic for the patient's cancer. Concentrations of glucose, lactic acid, and/or ATP in the cell growth medium and/or cells in a well containing a cell sample can be determined, and the determination can be made in various ways, such as by enzymatic methods, luminescent methods, biochemical methods, immunoassay method, spectrophotometry method, electrochemical methods, and light emission methods.

The test or assay of the invention can be used for determining the most effective anticancer agents to be used in treating a specific cancer in both humans or animals regardless of the cancer or tumor tissue origin. It can also be used to determine the cancer's resistance to particular anticancer agents and provide an indication as to which anticancer agents should not be used in treating the specific cancer.

In some cases it may be desirable to provide control samples obtained from a patient's normal tissues. In general, normal tissues produce little lactic acid in human patients or in animals. Thus, the control sample of normal tissue, when tested for lactic acid production, should show little lactic acid production. If the patient's normal tissue shows significant lactic acid production, the test of the invention may not be appropriate for that patient, human or animal.

Whereas the invention is here described with reference to embodiments thereof presently contemplated as the best mode of carrying out the invention in actual practice, it is to be understood that various changes may be made in adapting the invention to different embodiments without departing from the broader inventive concepts disclosed herein and comprehended by the claims that follow.

1. A method of predicting effective anticancer agents for treating a particular cancer in a patient, comprising the steps of:
obtaining a plurality of samples of the cancerous tissue from the particular cancer to be treated in a patient;
determining the capacity of each tissue sample to convert glucose to lactic acid;
determining the capacity of a tissue sample to convert glucose to lactic acid in the presence of a particular anticancer agent;
whereby the particular anticancer agents that most inhibit the capacity of a sample to convert glucose to lactic acid are the anticancer agents indicated as the most promising as effective anticancer agents to administer to the patient for treatment of the particular cancer.

2. A method of predicting effective anticancer agents for treating a particular cancer in a patient according to claim 1, additionally including the steps of determining the ATP level associated with each tissue sample, and determining the ATP level of the sample in the presence of the particular anticancer agent, whereby the particular anticancer agents of the plurality of possible anticancer agents resulting in the most reduced ATP levels are the anticancer agents indicated as the most promising as effective anticancer agents to administer to the patient for treatment of the particular cancer.

3. A method of predicting effective anticancer agents for treating a particular cancer in a patient according to claim 1, additionally including for those anticancer agents indicated as the most promising as effective anticancer agents after the steps of claim 1, the steps of determining the ATP level associated with respective tissue samples, and determining the ATP level of the respective sample in the presence of a particular indicated anticancer agent, whereby the particular anticancer agents of the indicated most promising anticancer agents resulting in the most reduced ATP levels are the anticancer agents indicated as the most promising as effective anticancer agents to administer to the patient for treatment of the particular cancer.

4. A method of predicting effective anticancer agents for treating a particular cancer in a patient, comprising the steps of:
obtaining a plurality of samples of the cancerous tissue from the particular cancer to be treated in a patient;
determining the capacity of each tissue sample to convert glucose to lactic acid;
placing each sample in the presence of one particular anticancer agent of a plurality of expected anticancer agents and determining the capacity of each respective sample to convert glucose to lactic acid in the presence of the particular anticancer agent;
whereby the particular anticancer agents that most inhibit the capacity of a sample to convert glucose to lactic acid are the anticancer agents indicated as the most promising as effective anticancer agents to administer to the patient for treatment of the particular cancer.

5. A method of predicting effective anticancer agents for treating a particular cancer in a patient, comprising the steps of:
obtaining a plurality of samples of the cancerous tissue from the particular cancer to be treated in a patient;
placing each of the plurality of tissue samples in a respective separate container with a cell culture containing glucose and determining the capacity of each separate tissue sample to convert glucose to lactic acid;
after determining the capacity of each separate tissue sample to convert glucose to lactic acid, adding a different particular anticancer agent of a plurality of possible anticancer agents to a different container of the plurality of containers containing the tissue samples and determining the capacity of each sample to convert glucose to lactic acid in the presence of the particular anticancer agent added thereto;
whereby the particular anticancer agents of the plurality of possible anticancer agents that most inhibit the capacity of a sample to convert glucose to lactic acid are the anticancer agents indicated as the most promising as effective anticancer agents to administer to the patient for treatment of the particular cancer.

6. A method of predicting effective anticancer agents for treating a particular cancer in a patient according to claim 5, additionally including the steps of determining the ATP levels associated with each tissue sample in a container before addition of the anticancer agent to the container; and determining the ATP levels of the sample after addition of the anticancer agent, whereby the particular anticancer agents of the plurality of possible anticancer agents resulting in the most reduced ATP levels are the anticancer agents indicated as the most promising as effective anticancer agents to administer to the patient for treatment of the particular cancer.

7. A method of predicting effective anticancer agents for treating a particular cancer in a patient according to claim 6, whereby the particular anticancer agents of the plurality of possible anticancer agents that most inhibit the capacity of a sample to convert glucose to lactic acid in combination with the most reduced ATP levels are the anticancer agents indicated as the most promising as effective anticancer agents to administer to the patient for treatment of the particular cancer.

8. A method of predicting effective anticancer agents for treating a particular cancer in a patient according to claim 5, wherein the individual containers are individual wells of multi-well plate.

9. A method of predicting a particular cancer's resistance to a particular anticancer agent available for treating the particular cancer in a patient, comprising the steps of:
obtaining a sample of the cancerous tissue from the particular cancer to be treated in a patient;
determining the capacity of the tissue sample to convert glucose to lactic acid;
determining the capacity of the tissue sample to convert glucose to lactic acid in the presence of the particular anticancer agent;
whereby a failure of the particular anticancer agent to substantially inhibit the capacity of the tissue sample to convert glucose to lactic acid is an indication of the particular cancer's resistance to the particular anticancer agent.

10. A method of predicting a particular cancer's resistance to a particular anticancer agent available for treating the particular cancer in a patient according to claim 9, additionally including the steps of determining the ATP level associated with the tissue sample before addition of the anticancer agent to the tissue sample; and determining the ATP levels of the sample after addition of the anticancer agent, whereby a lack of the particular anticancer agent to substantially reduce the ATP levels of the tissue sample is an indication of the particular cancer's resistance to the particular anticancer agent.
